(19) United States
(12) Patent Application Publication
(10) Pub. No.: US 2023/0117075 A1
(43) Pub. Date: Apr. 20, 2023

(54) TINTING SYSTEM FOR GOGGLES

(71) Applicant: Leroy Eifler, II, Litchfield Park, AZ (US)

(72) Inventor: Leroy Eifler, II, Litchfield Park, AZ (US)

(21) Appl. No.: 17/505,888

(22) Filed: Oct. 20, 2021

(51) Int. Cl. A61F 9/02; A42B 3/26

(52) U.S. Cl. CPC A61F 9/025; A61F 9/022; A42B 3/26

(57) ABSTRACT

A tinting system for goggles for use when riding a dirt bike incorporating a combination of clear transparent layers of material and tinted layers of material upon the front surface of the goggles that are adapted to be torn off one at a time in a fast and efficient way to rid the goggles of dirt and grime and to adjust the amount of tint the goggles provide for any given lighting condition.

### CROSS-REFERENCE TO RELATED APPLICATION

There are no related applications incorporated herein by reference.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever. 37 CFR 1.71(d).

### BACKGROUND OF THE INVENTION

### 1. Field of the Invention

This invention relates generally to a tinting system for goggles for use when riding a dirt bike and allows a user to quickly peel off layers of material to adjust the amount of tint the goggles offer for changing lighting conditions.

### 2. Description of the Prior Art

Dirt bike riders typically wear goggles to protect their eyes. When in use, goggles typically become covered with dirt and grime. Over the past 10 to 15 years it has become common practice to attached layers of transparent material over the front of the goggles that are adapted to be torn off one at a time as a fast and efficient way to rid the goggles of the dirt and grime. Goggles are also design with tinting infused therein to protect a riders' eyes from bright lights and glare. The problem occurs during changing lighting conditions. Currently there is no way to change the tinting of the goggles a rider is using to adjust to the changing lighting conditions. A rider usually changes their goggles for the changing lighting conditions. However, the rider would need to stop, take their hands off the handle bars of the dirt bike, locate, adjust, and properly place the new set of goggles upon their head before resuming. In racing scenarios, this is time consuming. Furthermore, other appropriate sets of goggles may not be available at the time the lighting conditions are changing. Accordingly, the present invention overcomes the disadvantages associated with the prior art by providing a combination of clear transparent layers of material and tinted layers of material upon the front surface of the goggles that are adapted to be torn off one at a time as a fast and efficient way to rid the goggles of dirt and grime and to adjust the amount of tint the goggles provide for any given lighting condition.

### BRIEF SUMMARY OF THE INVENTION

In view of the foregoing disadvantages inherent in the known types of goggles and tinting systems or like in the prior art, the present invention provides a tinting system for goggles for use when riding a dirt bike and allows a user to quickly peel off layers of material to adjust the amount of tint the goggles offer for changing lighting conditions. As such, the general purpose of the present invention, which will be described subsequently in greater detail, is to provide a system for goggles that not only is adapted to quickly rid the goggles of dirt and grime, but also adapted to allow the adjustment of the amount of tint the goggles provide for any given lighting condition.

There has thus been outlined, rather broadly, the more important features of the invention in order that the detailed description thereof that follows may be better understood and in order that the present contribution to the art may be better appreciated.

Numerous objects, features and advantages of the present invention will be readily apparent to those of ordinary skill in the art upon a reading of the following detailed description of presently preferred, but nonetheless illustrative, embodiments of the present invention when taken in conjunction with the accompanying drawings. The invention is capable of other embodiments and of being practiced and carried out in various ways. Also, it is to be understood that the phraseology and terminology employed herein are for the purpose of descriptions and should not be regarded as limiting

### BRIEF DESCRIPTION OF THE DRAWINGS

The figures which accompany the written portion of this specification illustrate embodiments according to the teachings of the present invention.

FIG. 1 shows a perspective view of a pair of goggles and the tinting system for goggles according to the preferred embodiment of the present invention.

FIG. 2 shows a perspective view of a pair of goggles including the tinting system for goggles according to the preferred embodiment of the present invention of FIG. 1.

FIG. 3 shows a perspective view of the combination of clear transparent layers of material and tinted layers of material of the tinting system for goggles according to the preferred embodiment of the present invention of FIG. 1.

The various embodiments of the present invention will hereinafter be described in conjunction with the appended drawings.

### DETAILED DESCRIPTION

The embodiments of the present disclosure described below are not intended to be exhaustive or to limit the disclosure to the precise forms disclosed in the following detailed description. Rather, the embodiments are chosen and described so that others skilled in the art may appreciate and understand the principles and practices of the present disclosure.

The following embodiments and the accompanying drawings, which are incorporated into and form part of this disclosure, illustrate embodiments of the invention and together with the description, serve to explain the principles of the invention. To the accomplishment of the foregoing and related ends, certain illustrative aspects of the invention are described herein in connection with the following description and the annexed drawings. These aspects are indicative, however, of but a few of the various ways in which the principles of the invention can be employed and the subject invention is intended to include all such aspects and their equivalents. Other advantages and novel features of the invention will become apparent from the following detailed description of the invention when considered in conjunction with the drawings.

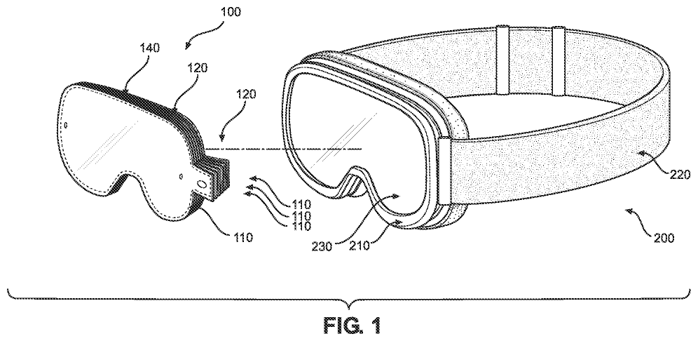

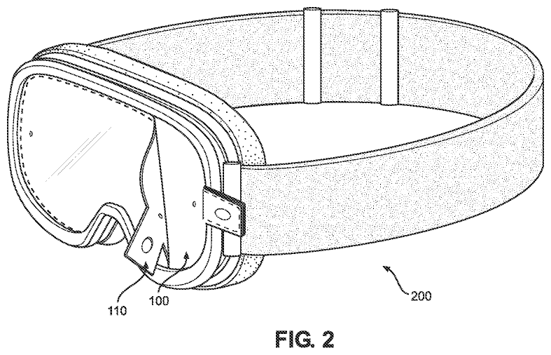

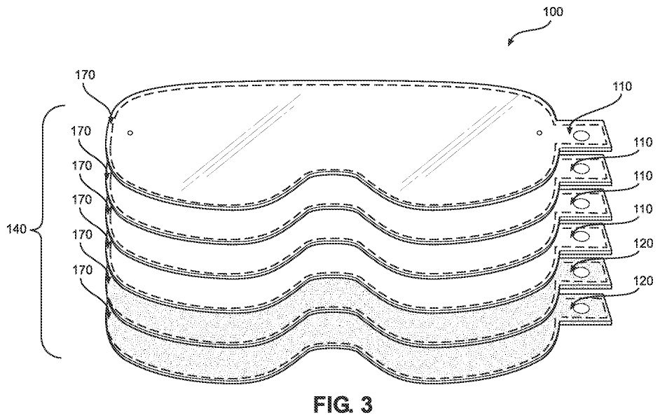

Turning now descriptively to drawing, referring to FIGS. 1-3, the present invention discloses a tinting system 100 for goggles 200, wherein the goggles 200 typically including a frame 210; a strap 220 attached to the frame and is adapted to allow a user to releasably place the goggles adjacent to their eyes; and a transparent lens 230 attached to the frame. The tinting system 100 comprises a plurality of clear transparent layers of material 110; and a plurality of tinted layers of material 120, wherein the plurality of clear transparent layers of material and the plurality of tinted layers of material are releasably attachable to one another, wherein the plurality of clear transparent layers of material and the plurality of tinted layers of material are adapted to be placed one on top of the other forming a stack 140, wherein the stack is sized, shaped, and adapted to be releasably attached to the front of the transparent lens 230 of the goggles 200 for use in enhancing the vision of a user while using the goggles, and wherein the plurality of clear transparent layers of material and the plurality of tinted layers of material of the stack are adapted to be peeled off from the stack one layer at a time.

The stack 140 may comprise a plurality of the clear transparent layers of material 110 placed on top of a plurality of the tinted layers of material 120, such that when in use the user can peel off the plurality of clear transparent layers of material one at a time from the top of the stack downwards, and wherein the user can peel off the plurality of tinted layers of material one at a time from the bottom of the stack upwards. As such, a user can select a predetermined number of layers of the plurality of clear transparent layers of material and the plurality of tinted layers of material to be used to form the stack to adapt the goggles to varying lighting conditions. In a preferred embodiment, there are four clear transparent layers of material 110 and two tinted layers of material 120.

The plurality of clear transparent layers of material 110 and the tinted layers of material 120 include an adhesive 170 on a portion of their respective surfaces adapted to allow respective layers of clear transparent and tinted material to be releasably attached to one another. The plurality of clear transparent layers of material and said plurality of tinted layers of material are formed having identical shapes, wherein the shape is adapted to match the shape of the lens of the goggles. The plurality of clear transparent layers of material and said plurality of tinted layers of material are formed from a flexible film chosen from a list of materials consisting of plastic, polyester, and cellophane.

The plurality of tinted layers of material 120 may also each be formed having a differing quantity of tinting infused therein, such that said user can more efficiently control the amount of tinting to be placed within said stack.

Although specific embodiments have been illustrated and described herein, it will be appreciated by those of ordinary skill in the art that any arrangement, which is calculated to achieve the same purpose, may be substituted for the specific embodiment shown. This application is intended to cover any adaptations or variations of the present invention.

Although the invention has been explained in relation to its preferred embodiment, it is to be understood that many other possible modifications and variations can be made without departing from the spirit and scope of the invention.

What is claimed is:

1. A tinting system for goggles, comprising:
a plurality of clear transparent layers of material; and
a plurality of tinted layers of material;
wherein said plurality of clear transparent layers of material and said plurality of tinted layers of material are releasably attachable to one another;
wherein said plurality of clear transparent layers of material and said plurality of tinted layers of material are adapted to be placed one on top of the other forming a stack;
wherein said stack is sized, shaped, and adapted to be releasably attached to the front of the lenses of goggles for use in enhancing the vision of a user while using said goggles; and
wherein said plurality of clear transparent layers of material and said plurality of tinted layers of material of said stack are adapted to be peeled off from the stack one layer at a time.

2. The tinting system for goggles of claim 1, wherein said stack comprises a plurality of said clear transparent layers of material placed on top of a plurality of said tinted layers of material, such that when in use said user can peel off said plurality of clear transparent layers of material one at a time from the top of said stack downwards, and wherein said user can peel off said plurality of tinted layers of material one at a time from the bottom of said stack upwards.

3. The tinting system for goggles of claim 2, wherein said user can predetermine the number of layers of said plurality of clear transparent layers of material and said plurality of tinted layers of material to be used to form said stack to adapt said goggles to varying lighting conditions.

4. The tinting system for goggles of claim 3, wherein there are four clear transparent layers of material and two tinted layers of material.

5. The tinting system for goggles of claim 1, wherein said plurality of said clear transparent layers of material and said tinted layers of material include an adhesive on a portion of their respective surfaces adapted to allow respective layers of clear transparent and tinted material to be releasably attached to one another.

6. The tinting system for goggles of claim 1, wherein said plurality of clear transparent layers of material and said plurality of tinted layers of material are formed having identical shapes.

7. The tinting system for goggles of claim 6, wherein said shape of said plurality of clear transparent and tinted layers of material is adapted to match the shape of lens of said goggles.

8. The tinting system for goggles of claim 1, wherein said plurality of clear transparent layers of material and said plurality of tinted layers of material are formed from a flexible film.

9. The tinting system for goggles of claim 8, wherein said flexible film is formed from a material chosen from a list of materials consisting of plastic, polyester, and cellophane.

10. The tinting system for goggles of claim 1, wherein said plurality of tinted layers of material are each formed having a differing quantity of tinting infused therein, such that said user can more efficiently control the amount of tinting to be placed within said stack.

11. A combination of a goggles and a tinting system, comprising:
goggles including:
a frame;
a strap;
wherein said strap is attached to said frame and is adapted to allow a user to releasably place said goggles adjacent to their eyes; and
a transparent lens;
wherein said transparent lens is attached to said frame; and
a tinting system comprising:
a plurality of clear transparent layers of material; and
a plurality of tinted layers of material;
wherein said plurality of clear transparent layers of material and said plurality of tinted layers of material are releasably attachable to one another;
wherein said plurality of clear transparent layers of material and said plurality of tinted layers of material are adapted to be placed one on top of the other forming a stack;
wherein said stack is sized, shaped, and adapted to be releasably attached to the front of said transparent lens of said goggles for use in enhancing the vision of a user while using said goggles; and
wherein said plurality of clear transparent layers of material and said plurality of tinted layers of material of said stack are adapted to be peeled off from the stack one layer at a time.

12. The combination of claim 11, wherein said stack comprises a plurality of said clear transparent layers of material placed on top of a plurality of said tinted layers of material, such that when in use said user can peel off said plurality of clear transparent layers of material one at a time from the top of said stack downwards, and wherein said user can peel off said plurality of tinted layers of material one at a time from the bottom of said stack upwards.

13. The combination of claim 12, wherein said user can predetermine the number of layers of said plurality of clear transparent layers of material and said plurality of tinted layers of material to be used to form said stack to adapt said goggles to varying lighting conditions.

14. The combination of claim 13, wherein there are four clear transparent layers of material and two tinted layers of material.

15. The combination of claim 11, wherein said plurality of said clear transparent layers of material and said tinted layers of material include an adhesive on a portion of their respective surfaces adapted to allow respective layers of clear transparent and tinted material to be releasably attached to one another.

16. The combination of claim 1, wherein said plurality of clear transparent layers of material and said plurality of tinted layers of material are formed having identical shapes.

17. The combination of claim 16, wherein said shape of said plurality of clear transparent and tinted layers of material is adapted to match the shape of said lens of said goggles.

18. The combination of claim 11, wherein said plurality of clear transparent layers of material and said plurality of tinted layers of material are formed from a flexible film.

19. The combination of claim 18, wherein said flexible film is formed from a material chosen from a list of materials consisting of plastic, polyester, and cellophane.

20. The combination of claim 11, wherein said plurality of tinted layers of material are each formed having a differing quantity of tinting infused therein, such that said user can more efficiently control the amount of tinting to be placed within said stack.
